(19) United States
(12) Patent Application Publication
(10) Pub. No.: US 2012/0265219 A1
(43) Pub. Date: Oct. 18, 2012

(54) ARTHROSCOPIC TECHNIQUE FOR LOAD SHARING WITH PATCH AND SUTURE ASSEMBLY

(76) Inventors: Jamal RUSHDY, Eden Prairie, MN (US); Justin C. ANDERSON, Minnetonka, MN (US); Kevin OHASHI, Jamaica Plain, MA (US); Heber CROCKETT, Kearney, NE (US); Gary GARTSMAN, Houston, TX (US); Mark SCHAMBLIN, Bakersfield, CA (US)

(21) Appl. No.: 13/292,067

(22) Filed: Nov. 8, 2011

Related U.S. Application Data
(60) Provisional application No. 61/411,428, filed on Nov. 8, 2010.

(51) Int. Cl. A61B 17/062

(52) U.S. Cl. 606/139

(57) ABSTRACT

A method according to embodiments of the present invention includes attaching an assembly of suture and patch across a tendon to be repaired, for example a rotator cuff, and adjusting the tension of the suture and patch assembly independent of the tension of the repaired tendon.

### CROSS-REFERENCE TO RELATED APPLICATION

This application claims the benefit of U.S. Provisional Patent Application Ser. No. 61/411,428 filed on Nov. 8, 2010, which is incorporated by reference herein in its entirety for all purposes.

### TECHNICAL FIELD

Embodiments of the present invention relate to constructs and methods for load sharing between a sutured patch and a repaired tissue.

### BACKGROUND

In repairing tissue such as tendons or ligaments, suturing the torn end of the tissue to bone and/or to bone anchors involves placing the full load experienced by the tissue across the suture-to-tissue interface. This can cause challenges for healing and/or tissue performance, particularly at a time when the tissue is weak and before any healing has occurred.

### SUMMARY

A method for implementing load sharing among a tissue to be repaired and a patch according to embodiments of the present invention includes arthroscopically forming a first bone tunnel, the first bone tunnel continuous from a first end to a second end, arthroscopically forming a second bone tunnel, the second bone tunnel continuous from a first end to a second end, arthroscopically inserting a first suture loop through the first bone tunnel into the first end and out of the second end, the first suture loop formed of a first suture, arthroscopically inserting a second suture loop through the second bone tunnel into the first end and out of the second end, the second suture loop formed of a second suture, passing the first suture loop through a patch, wherein the patch is a synthetic or biologic biocompatible tissue repair patch, passing the second suture loop through the patch, and passing a third suture through the first and second suture loops. Such methods may further include passing a first end of the third suture through the patch, and passing a second end of the third suture through the patch. The first and second ends of the third suture may be crossed over the patch before passing the first and second ends of the third suture through the patch. Also, a fourth suture may be passed through the first and second suture loops.

Such methods may further include passing a first end of the third suture through the patch, passing a second end of the third suture through the patch, passing a first end of the fourth suture through the patch, and passing a second end of the fourth suture through the patch. The first end of the third suture may be passed through the patch through a first hole, and the second end of the third suture may be passed through the patch through a second hole separated from the first hole. Such methods may further include passing the first end of the fourth suture through the first hole, and passing the second end of the fourth suture through the second hole, and/or crossing the first and second ends of the fourth suture before passing the first end of the fourth suture through the first hole and the second end of the fourth suture through the second hole.

Such methods according to embodiments of the present invention may include applying a first self-reinforcing stitch through the patch with the first end of the third suture and the first end of the fourth suture, and applying a second self-reinforcing stitch through the patch with the second end of the third suture and the second end of the fourth suture. The patch may be tensioned by tensioning the first and second ends of the third and fourth sutures. The first suture may be tied to the second suture across the bone to form a suture bridge, according to embodiments of the present invention. Such methods may further include tying the first ends of the third and fourth sutures to the suture bridge, and tying the second ends of the third and fourth sutures to the suture bridge.

A method for implementing load sharing among a tissue to be repaired and a patch according to embodiments of the present invention includes passing, arthroscopically, a first suture loop of a first suture through tissue to be repaired, passing the first suture loop through a patch, wherein the patch is a synthetic or biologic biocompatible surgical repair patch, passing, arthroscopically, a second suture loop of a second suture through the tissue, passing the second suture loop through the patch, passing a third suture through the first and second suture loops, passing a first end of the third suture through the patch, passing a second end of the third suture through the patch, attaching, arthroscopically, the first suture to a bone under the tissue, attaching, arthroscopically, the second suture to the bone under the tissue, tensioning the third suture and the first and second suture loops over the patch, and anchoring the first and second ends of the third suture to the bone. Such methods may further include passing a fourth suture through the first and second suture loops, passing a first end of the fourth suture through the patch, passing a second end of the fourth suture through the patch, and attaching the first and second ends of the fourth suture to the bone.

According to some embodiments, anchoring the first and second ends of the third suture to the bone includes attaching at least one of the first and second ends of the third suture to a ring cinch suture anchor, the method further including adjusting a tension of the at least one of the first and second ends of the third suture independently of a tension of the tissue. According to some embodiments of the present invention, the surgeon may make a first tension adjustment to the at least one of the first and second ends of the third suture, observe the tension of the tissue, and then make a second tension adjustment to the at least one of the first and second ends of the third suture based on the observation.

A method for implementing load sharing among rotator cuff tissue to be repaired and a patch and suture assembly according to embodiments of the present invention includes forming the patch and suture assembly from a patch and suture to permit tension applied to the suture to be transmitted through the patch, anchoring the patch and suture assembly medially through the rotator cuff tissue, adjusting a tension of the patch and suture assembly independent of a tension of the rotator cuff tissue, and anchoring the patch and suture assembly laterally at the adjusted tension. According to some embodiments, anchoring the patch and suture assembly laterally includes installing a knotless or tensionable-type suture anchor, and adjusting the tension of the patch and suture assembly includes pulling the suture through the knotless or tensionable-type suture anchor. In some cases, anchoring the patch and suture assembly medially and laterally includes tying the patch and suture assembly to one or more transosseous bone tunnels. Anchoring the patch and suture assembly may include tying the patch and suture assembly to one or more transosseous bone tunnels without the use of bone anchors, according to embodiments of the present invention.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a posterior-lateral anatomical view of an anatomical human shoulder.

FIG. 2 is a posterior-lateral anatomical view of a left human shoulder with a torn supraspinatus tendon.

FIG. 3 is a posterior anatomical view of a right human shoulder with a torn supraspinatus tendon.

FIG. 4 is a posterior-lateral anatomical view of a left human shoulder with a prior art arthroscopic repair of a torn supraspinatus tendon.

FIG. 5 is a posterior-lateral anatomical view of a left human shoulder with a prior art patch repair of a torn supraspinatus tendon.

FIG. 6 illustrates a placement of suture loops through transosseous bone tunnels and tissue, according to embodiments of the present invention.

FIG. 7 illustrates placement of suture loops through a patch, according to embodiments of the present invention.

FIG. 8 illustrates placement of free sutures through suture loops, according to embodiments of the present invention.

FIG. 9 illustrates further placement of free sutures through the patch, according to embodiments of the present invention.

FIG. 10 illustrates further placement of free suture ends through the patch, according to embodiments of the present invention.

FIG. 11 illustrates arthroscopic insertion of the patch and suture assembly, according to embodiments of the present invention.

FIG. 12 illustrates arthroscopic insertion of the patch and suture assembly, according to embodiments of the present invention.

FIG. 13 illustrates arthroscopic insertion of the patch and suture assembly, according to embodiments of the present invention.

FIG. 14 illustrates a lateral tie off of sutures, according to embodiments of the present invention.

FIG. 15 illustrates a further tying of the patch sutures to the bone tunnel sutures, according to embodiments of the present invention.

FIG. 16 illustrates an alternative suture placement in a patch, according to embodiments of the present invention.

FIG. 17 illustrates anchoring of the patch of FIG. 17, according to embodiments of the present invention.

FIG. 18 illustrates an alternative suture placement in a patch, according to embodiments of the present invention.

FIG. 19 illustrates further suture placement for the patch of FIG. 19, according to embodiments of the present invention.

FIG. 20 illustrates formation of a self-reinforcing stitch for the patch of FIG. 19, according to embodiments of the present invention.

FIG. 21 illustrates a tie off and anchoring for the patch of FIG. 19, according to embodiments of the present invention.

FIG. 22 illustrates an enlarged view of an exemplary Modified Mason-Allen stitch, according to embodiments of the present invention.

FIG. 23 illustrates an alternative suture placement in a patch, according to embodiments of the present invention.

FIG. 24 illustrates the alternative suture placement in the patch of FIG. 24, according to embodiments of the present invention.

FIG. 25 illustrates a patch applied with an alternative technique for arthroscopic repair of a rotator cuff tear, according to embodiments of the present invention.

FIG. 26 illustrates a patch applied with yet another alternative technique for arthroscopic repair of a rotator cuff tear, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Embodiments of the present method and apparatus can be used to repair and reconstruct torn ligaments and tendons in a variety of locations of the body. The rotator cuff muscles were selected for the exemplary embodiments because of the complexity of the human shoulder. It will be appreciated that the methods and apparatus according to embodiments of the present invention may have many other possible applications.

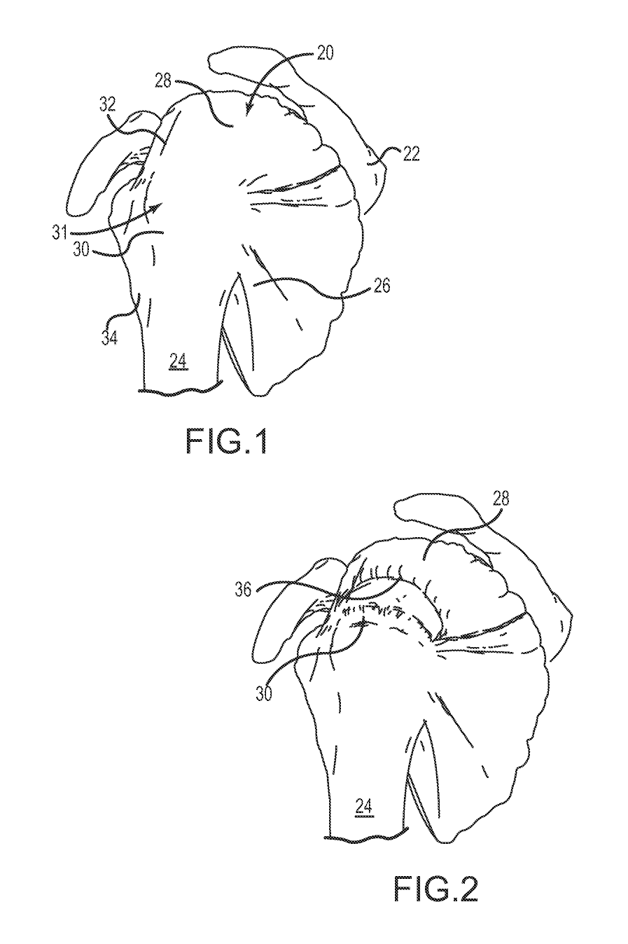

FIGS. 1-5 are taken from U.S. Patent Application Publication Number 2008/0188936, published on Aug. 7, 2008, which is incorporated by reference herein in its entirety. As illustrated in FIG. 1, the rotator cuff 20 is the complex of four muscles that arise from the scapula 22 and whose tendons blend in with the subjacent capsule as they attach to the tuberosities of the humerus 24. The subscapularis 26 arises from the anterior aspect of the scapula 20 and attaches over much of the lesser tuberosity. The supraspinatus muscle 28 arises from the supraspinatus fossa of the posterior scapula, passes beneath the acromion and the acromioclavicular joint, and attaches to the superior aspect of the greater tuberosity 30. The infraspinatus muscle 32 arises from the infraspinous fossa of the posterior scapula and attaches to the posterolateral aspect of the greater tuberosity 30. The teres minor 34 arises from the lower lateral aspect of the scapula 20 and attaches to the lower aspect of the greater tuberosity 30. Proper functioning of the rotator, 3 to 4 millimeters thick, depends on the fundamental centering and stabilizing role of the humeral head 31 with respect to sliding action during anterior and lateral lifting and rotation movements of the arm.

The insertion of these tendons as a continuous cuff 20 around the humeral head 31 permits the cuff muscles to provide an infinite variety of moments to rotate the humerus 24 and to oppose unwanted components of the deltoid and pectoralis muscle forces. The insertion of the infraspinatus 32 overlaps that of the supraspinatus 28 to some extent. Each of the other tendons 26, 34 also interlaces its fibers to some extent with its neighbor's tendons. The tendons splay out and interdigitate to form a common continuous insertion on the humerus 24. The biceps tendon is ensheathed by interwoven fibers derived from the subscapularis and supraspinatus.

The mechanics of the rotator cuff 20 is complex. The cuff muscles 20 rotate the humerus 24 with respect to the scapula 22, compress the humeral head 31 into the glenoid fossa providing a critical stabilizing mechanism to the shoulder (known as concavity compression), and provide muscular balance. The supraspinatus and infraspinatus provide forty-five percent of abduction and ninety percent of external rotation strength. The supraspinatus and deltoid muscles are equally responsible for producing torque about the shoulder joint in the functional planes of motion.

The rotator cuff muscles 20 are critical elements of this shoulder muscle balance equation. The human shoulder has no fixed axis. In a specified position, activation of a muscle creates a unique set of rotational moments. For example, the anterior deltoid can exert moments in forward elevation, internal rotation, and cross-body movement. If forward elevation is to occur without rotation, the cross-body and internal rotation moments of this muscle must be neutralized by other muscles, such as the posterior deltoid and infraspinatus. As another example, use of the latissimus dorsi in a movement of pure internal rotation requires that its adduction moment be neutralized by the superior cuff and deltoid. Conversely, use of the latissimus in a movement of pure adduction requires that its internal rotation moment be neutralized by the posterior cuff and posterior deltoid muscles.

The timing and magnitude of these balancing muscle effects must be precisely coordinated to avoid unwanted directions of humeral motion. Thus the simplified view of muscles as isolated motors, or as members of force couples must give way to an understanding that all shoulder muscles function together in a precisely coordinated way—opposing muscles canceling out undesired elements leaving only the net torque necessary to produce the desired action.

By contrast, muscles in the knee generate torques primarily about a single axis of flexion-extension. If the quadriceps pull is a bit off-center, the knee still extends. Consequently, the human shoulder is a good tool to illustrate the present method and apparatus.

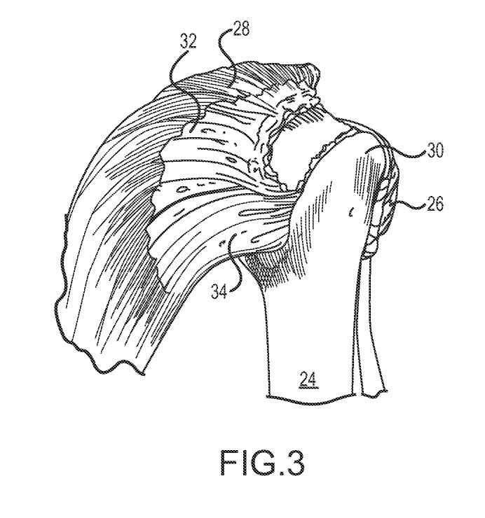

The suprasinatus 28 frequently tears away from the humerus 24 due to high stress activity or traumatic injury. FIG. 2 is an anterior view of a human left shoulder with a torn supraspinatus tendon 28. FIG. 3 is a posterior view of a human right shoulder with a torn supraspinatus tendon 28. The supraspinatus 28 has separated from the humerus 24 along its lateral edge 36 away from its attachment surface or “footprint” in the greater tuberosity 30.

Surgical repair is usually accomplished by reattaching the tendon back in apposition to the region of bone from which it tore. For the supraspinatus tendon 28 this attachment region, commonly called the “footprint”, occurs in a feature of the humerus 24 called the greater tuberosity 30. Repair is generally accomplished by sutured fixation of the tendon 28 directly to holes or tunnels created in the bone, or to anchoring devices embedded in the bone surface.

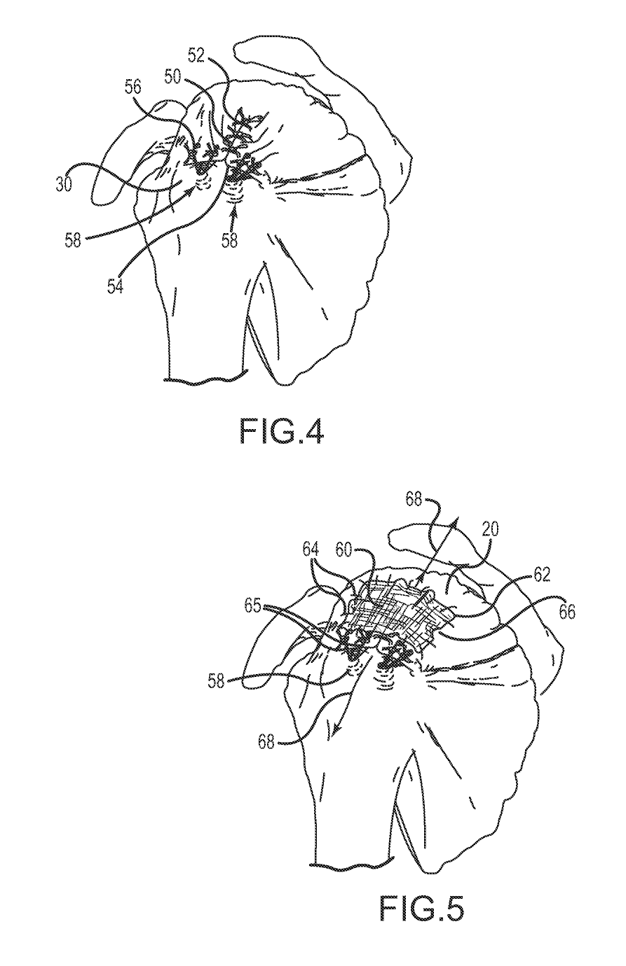

FIG. 4 shows a conventional arthroscopic repair of the torn suprasinatus tendon 28. The margins of the tear have been brought together at a convergence line 50 and closed by tendon-to-tendon stitches 52. The lateral edge 54 has been brought into apposition with the greater tuberosity 30 and secured in place through the use of four sutures 56 secured to two bone anchors 58 driven into the bone in the vicinity of the greater tuberosity 30. This state-of-the-art repair is subject to a 20-60% failure rate, primarily due to suture tear-out from poor quality tendon tissue.

FIG. 5 shows an improvement to the repair of FIG. 4 with the addition of a patch 60 augmenting the repair. The edges 62 of the substantially planar patch 60 are attached to the rotator cuff tendon 20 by sutures 64.

In spite of numerous recent advances in primary fixation repair, 20-60% of rotator cuff repairs fail, primarily due to suture tear-out in poor quality tendon tissue. A number of factors affect the quality of the tendon tissue to be repaired: Patient age, health, physical condition and lifestyle choices, as well as the time delay between when the injury occurred and surgery. These factors present the surgeon with tissue ranging from thick, strong healthy tissue that is easily moved into apposition with the footprint, to thin, friable, connective tissue attached to retracted or atrophied muscle. The case of retracted tissue presents a particular challenge to the surgeon since tendon of poor quality must be placed in tension to move it into apposition with the footprint, making it particularly prone to failure.

In an attempt to overcome these shortcomings, a class of biologically derived implant materials have been developed. These materials include allografts, (e.g. Wright Medical GraftJacket™ [Human Dermis]) and xenografts, (e.g. Depuy Restore™ (Porcine SIS), Arthrotek Cuff Patch™ [Porcine SIS], Stryker TissueMend™ [Fetal Bovine Dermis], Zimmer Permacol™ [Porcine Dermis], Pegasus Orthadapt™ [Equine Pericardium], Kensey Nash BioBlanket™ [Collagen], CryoLife ProPatch™ [Bovine Pericardium]). In addition to providing structural reinforcement, these materials are intended to repopulate the host ligament or tendon tissue with appropriate ligament or tendon cells as they are absorbed by the body.

Although the implant patch 70 of FIG. 5 helps to reinforce the rotator cuff tendon 20 and prevent suture pull-out with respect to the rotator cuff tendon 20, the implant patch 70 as implanted in FIG. 5 cannot be tensioned independently of the tendon 20 in a way permits customizable load sharing among the tendon 20 and the patch 70. Embodiments of the present invention permit load sharing and adjustment, arthroscopically, of the tension of the patch or graft, independent of the tension on the tendon or other repaired tissue.

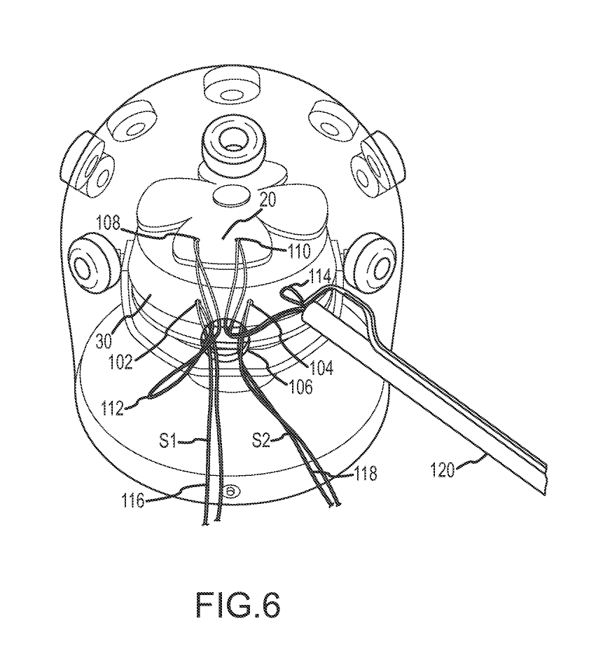

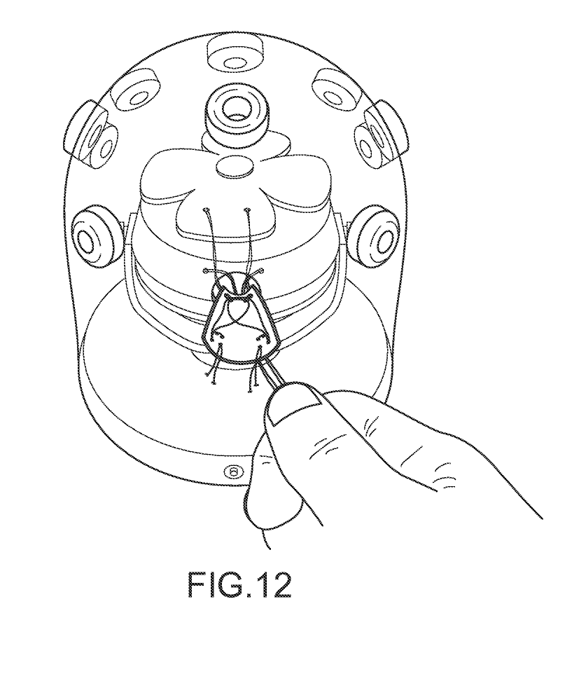

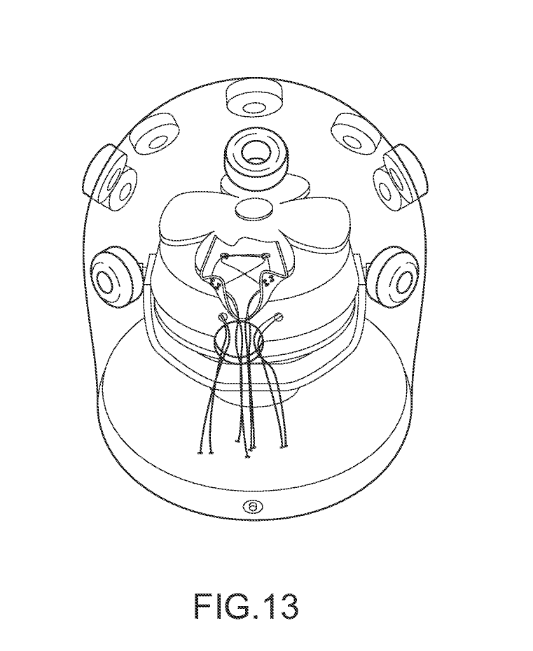

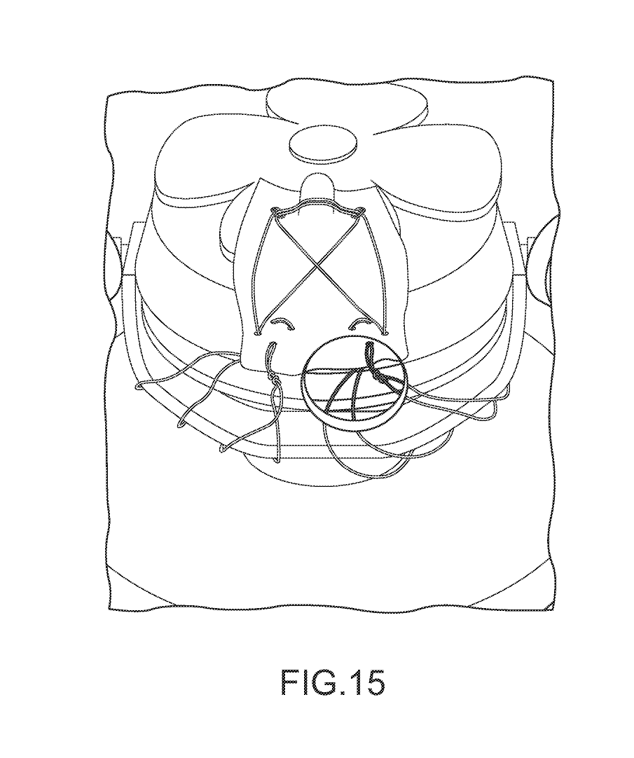

FIGS. 6-15 illustrate a method for implanting a patch in rotator cuff repair surgery so as to permit load sharing and independent tension adjustment of the sutures and patch with respect to the rotator cuff, according to embodiments of the present invention. As used herein, the term “patch” is used in its broadest sense to refer to an implant having one or more layers and an outer perimeter of any shape, and includes synthetic patches as well as biologic grafts, including allografts and xenografts. For example, a patch material may be made of a slow-absorbing, biologically benign material, such as Poly-4-hydroxybutyrate (e.g.Tephaflex™), poly(urethane urea) (Artelon™), surgical silk, polymers containing lactide, glycolide, caprolactone, trimethylene carbonate or dioxanone, or other materials, known to the art, having similar characteristics. In some cases, a patch may employ non-absorbable materials such as PTFE, Polyester, Polypropylene, Nylon, or other biocompatible, inert materials known to the art. In some embodiments, monofilaments may be used in combination with weaves, knits, braids, etc, to increase porosity for tissue in-growth and selective tensile strength along a load direction. The patch may also be constructed with or from allograft and/or xenograft materials, according to embodiments of the present invention.

FIG. 6 illustrates an arthroscopic shoulder model having an outer dome and a model shoulder on the inside of the dome. The model shoulder includes a medial rotator cuff 20 and a humeral head 30, which represent the corresponding anatomic structures in the human body. The outer dome includes one or more access ports 106, which represent an arthroscopic access port or cannula. In the illustrations, the steps and procedures achieved beneath the outer dome are performed arthroscopically, according to embodiments of the present invention.

A transosseous bone tunnel may be formed through the humeral bone 30 and through the tissue 20 using a transosseous tunneling system 120, for example the transosseous tunneling system described in U.S. Patent Application Publication No. 2011/0009867, published on Jan. 13, 2011, which is incorporated by reference herein in its entirety. The transosseous tunneling system forms one or more lateral holes 102, 104, as well as one or more medial holes 108, 110 which also extend through the rotator cuff 20 tissue, according to embodiments of the present invention. A first suture 51 may be folded to form a suture loop 112, which may also be passed lateral-to-medial through the bone tunnel and out the top of tissue 20 as shown, which may also be performed with the transosseous tunneling system. Similarly, a second suture S2 may be folded to form a suture loop 114, which may also be passed lateral-to-medial through the other bone tunnel and out the top of tissue 20 as shown. At this point, both suture loops 112, 114 and both free lateral ends 116, 118 of the first and second sutures S1, S2 may extend out of the arthroscopic access port 106, according to embodiments of the present invention.

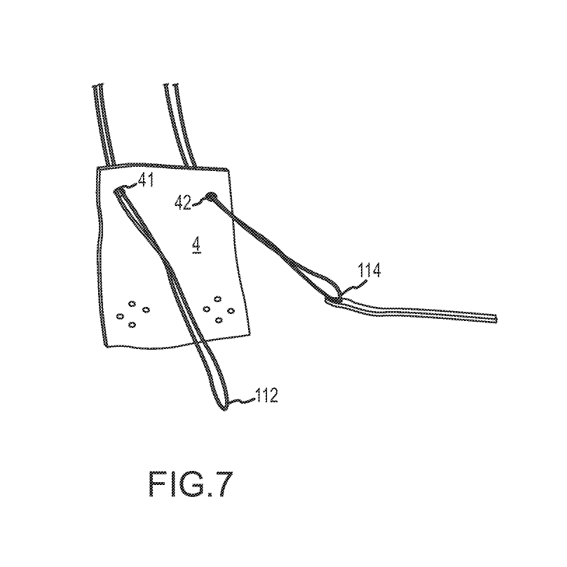

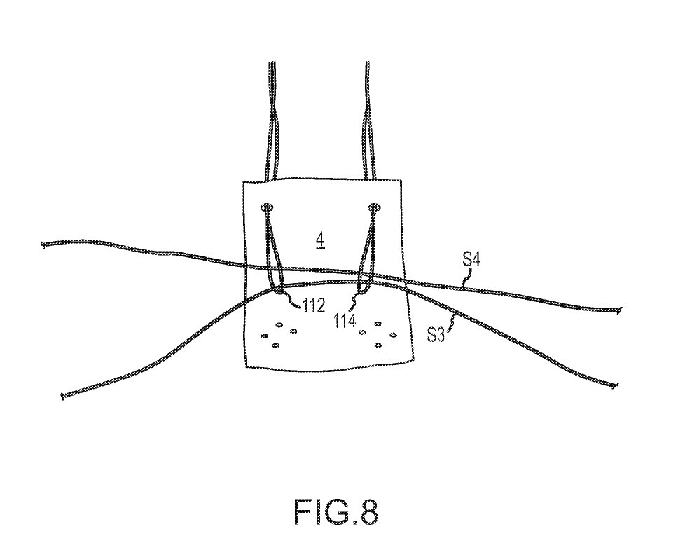

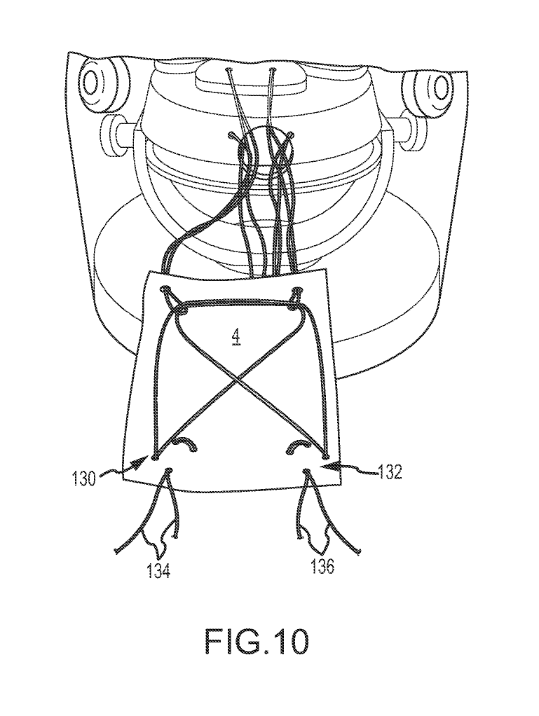

As shown in FIG. 7, the loop 112 is then passed through a patch 4 at location 41. The hole 41 may be pre-pierced, or may be formed with a needle or suture passer at the same time that loop 112 is passed through patch 4. Loop 114 may be passed through patch 4 in a similar fashion, at location 42, according to embodiments of the present invention. Locations 41, 42 may be along a medial edge of the patch 4, as illustrated in FIG. 7, according to embodiments of the present invention. FIG. 8 illustrates two additional free sutures S3, S4 being passed through both suture loops 112, 114, and FIG. 10 illustrates that the ends 122, 124 of one of the sutures S3 may be crossed and inserted back through the patch 4 along with the ends 126, 128 of the suture S4, according to embodiments of the present invention. As shown, the ends 122, 126 may be inserted through the same location (e.g. hole) 43, and the ends 124, 128 may be inserted through the same location (e.g. hole) 44, according to embodiments of the present invention.

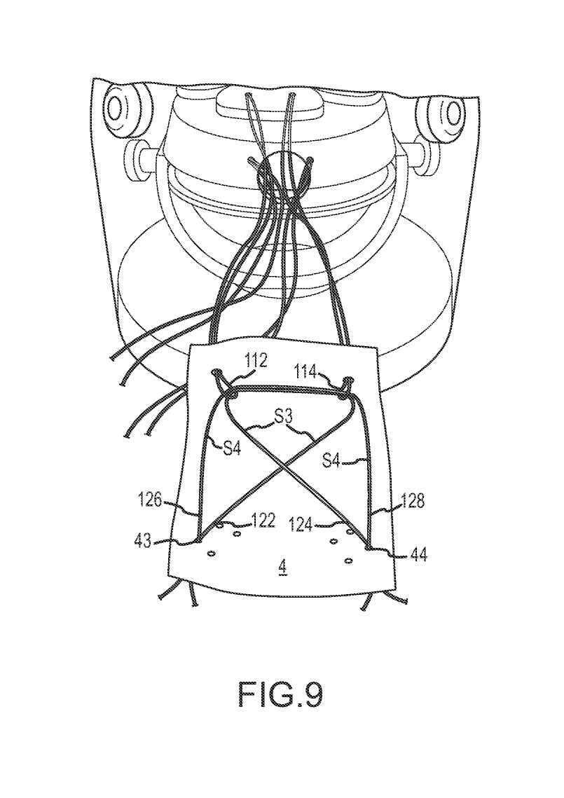

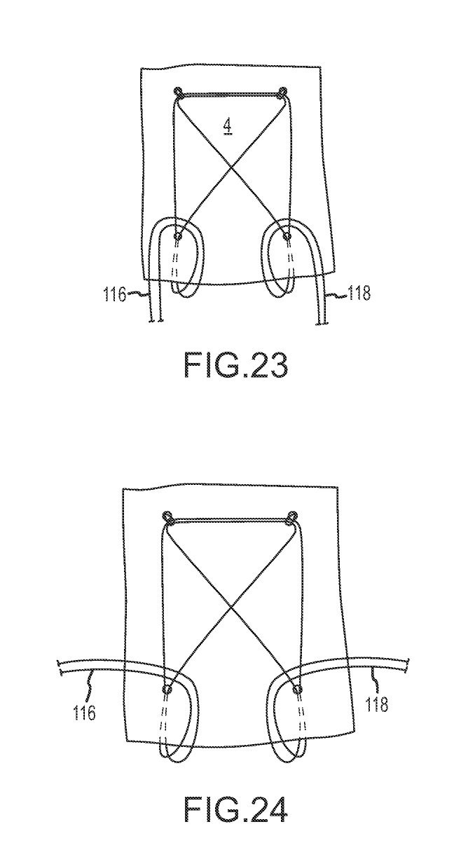

As shown in FIGS. 9 and 10, the ends 122, 126 of the sutures S3 and S4 may collectively be referred to as lateral end 134, and the ends 124, 128 of sutures S3 and S4 may collectively be referred to as lateral end 136. FIG. 10 illustrates that lateral end 134 and lateral end 136 may be stitched to the patch 4 with self-reinforcing stitches, for example a Modified Mason-Allen stitch as illustrated in FIGS. 10 and 23. Self-reinforcing stitches 130, 132 are stitches which permit tension applied to the lateral ends 134, 136 to be transmitted through the patch 4 rather than solely through the sutures, according to embodiments of the present invention. Self-reinforcing stitches may also be referred to as locking stitches, according to embodiments of the present invention. A self-reinforcing stitch is a stitch which involves passing the same suture end through the patch more than once at a different location, such that the suture passes through the patch or graft, then goes back through patch or graft in any direction one or more additional times, according to embodiments of the present invention.

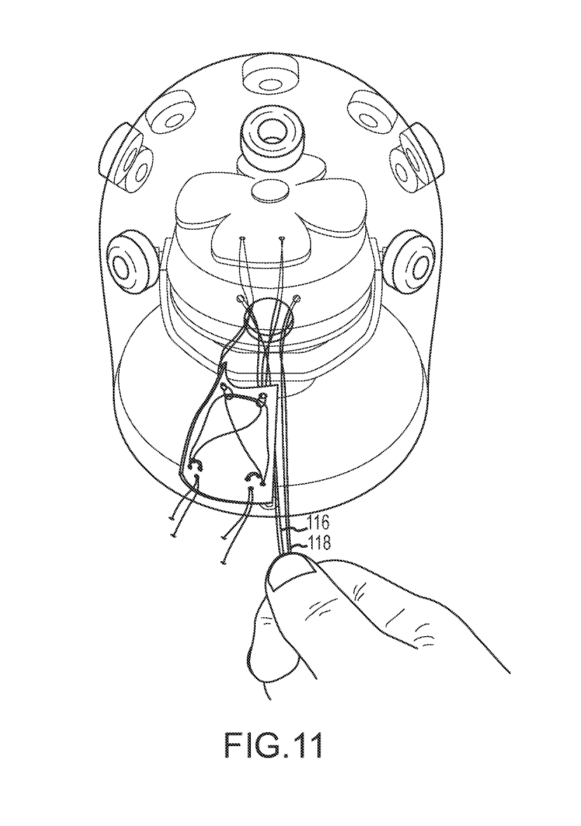

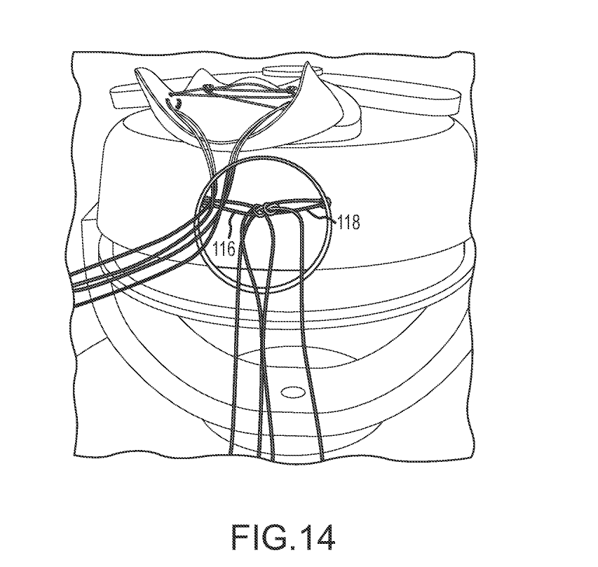

FIGS. 11-13 illustrate the arthroscopic insertion of the patch 4, according to embodiments of the present invention. By pulling on the free lateral ends 116, 118 of sutures S1 and S2, the patch 4 is pulled into the body. The patch 4 may be folded or bent, or may fold or bend of its own accord, as it passes through the arthroscopic access port, according to embodiments of the present invention. Once the patch 4 has achieved a desired placement, the surgeon may tie off the free lateral ends 116, 118 of the sutures S1 and S2, for example to each other, across the bone (between lateral holes 102 and 104) in an anterior-posterior direction, as shown in FIG. 14, according to embodiments of the present invention. As one variation to the procedure shown in FIG. 14, additional stabilization sutures may be applied on the anterior and posterior sides of the patch 4 outside the joint and then passed into the joint (e.g. with a spinal needle or the like) to enhance rotational control; as such, one would pull such additional stabilization sutures anterior-to-posterior instead of through the lateral port 106), according to embodiments of the present invention.

Finally, the surgeon may independently adjust the tension of the patch 4 and suture assembly before tying the free lateral ends 134, 136 to the free lateral ends 116, 118 (or to the suture bridge formed between holes 102 and 104 as shown in FIG. 14) to secure the tensioned patch 4 in place, according to embodiments of the present invention.

As shown in FIGS. 6-15, the patch 4 is prevented from migrating laterally by the tie-off across bone tunnels 102, 104. The sutures S1 and S2 may alternatively be anchored into the bone 30 with bone anchors, for example bone anchors applied directly through tissue 20; such anchors may permit knotless fixation or may permit the surgeons to tie the sutures S1, S2 to the anchors medially at the desired placement of patch 4. Such anchors may be placed medially, for example through locations 108, 110, according to embodiments of the present invention.

In fact, the patch 4 may be placed in order to share loads without the use of a transosseous tunneling system 120, according to embodiments of the present invention. According to such embodiments, in addition to the use of medial suture anchors, lateral suture anchors may also be used to permit the surgeon to independently adjust the tension on the patch 4. For example, the free lateral ends 134 and 136 (as shown in FIG. 11) may each be passed through a knotless suture anchor system anchored into the bone 30. Such a knotless suture fixation system may be similar to those described in U.S. Pat. No. 7,938,847, granted on May 10, 2011, which is incorporated by reference herein in its entirety. The use of such knotless suture fixation systems, for example a ring-cinch type anchor which permits suture to be pulled through the anchor in one direction but which grips the suture to prevent its loosening along another direction, permit the surgeon a high degree of control over the particular tension applied to patch 4. Using such a ring-cinch type anchor system also permits the surgeon to select different tension levels for each lateral end 134, 136, according to embodiments of the present invention. Such ring-cinch type anchors may also optionally be used for the medial anchors, according to embodiments of the present invention.

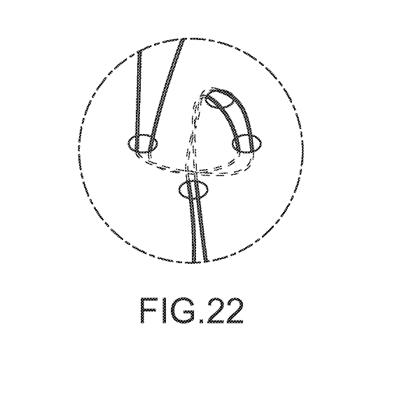

The use of a self-reinforcing stitch 130, 132 in the patch, for example as shown in FIGS. 10 and 22, permits tension applied to ends 134, 136 to be applied through the patch 4 rather than just along the sutures. Based on the disclosure provided herein, one of ordinary skill in the art will recognize numerous other self-reinforcing stitches that may be used to achieve a similar result. Without the use of the lateral self-reinforcing stitches 130, 132, when the lateral ends 134, 136 are anchored, the patch 4 acts as an overlay patch without significant or independently-adjustable load sharing, according to embodiments of the present invention.

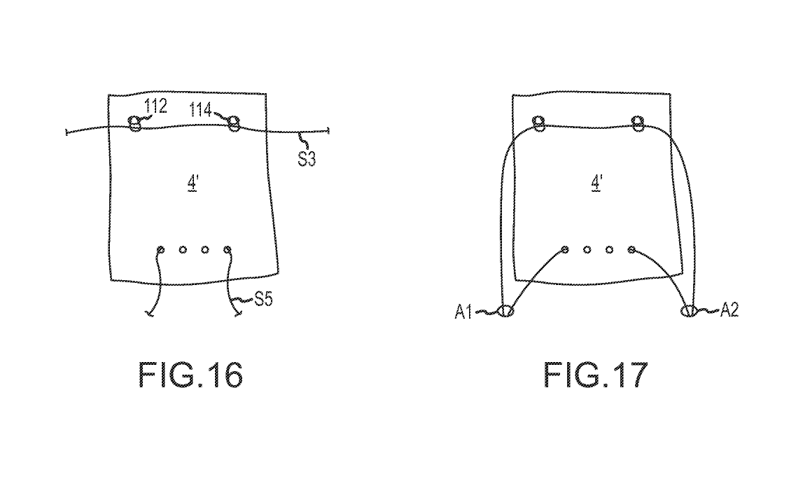

FIGS. 16 and 17 illustrate an alternative process for load sharing across a patch 4′, according to embodiments of the present invention. Similar to the methods described with respect to FIGS. 6-15, one suture S3 may be placed through loops 112 and 114 medially. A single suture S5 may be placed in the middle of patch 4′ or laterally on patch 4′ with a mattress stitch as shown in FIG. 16. One free end of suture S3 and one free end of suture S5 may be anchored laterally using anchor A1, and the other free end of suture S3 and the other free end of suture S5 may be anchored laterally using anchor A2, according to embodiments of the present invention. Anchors A1 and/or A2 may be knotless anchors, for example ring-cinch type anchors as described above, according to embodiments of the present invention. Alternatively, the free ends of sutures S3 and S5 may be tied off to the bone tunnel sutures S1 and S2 laterally, as shown in FIG. 14, according to embodiments of the present invention. One or more self-reinforcing stitches may also be used, for example at the lateral end of patch 4′, to improve load sharing, according to embodiments of the present invention.

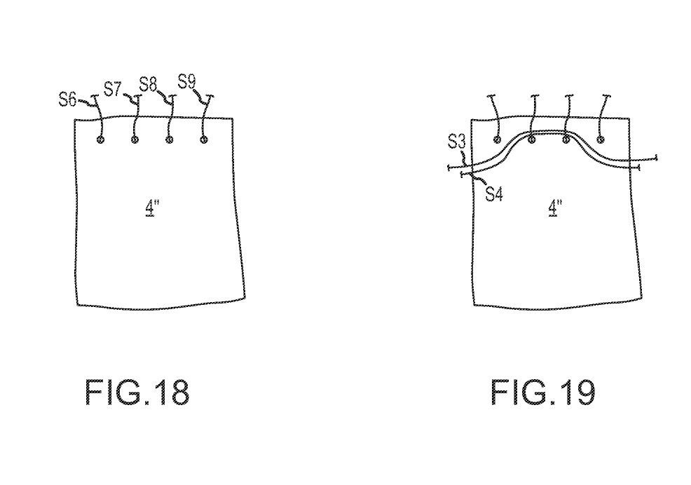

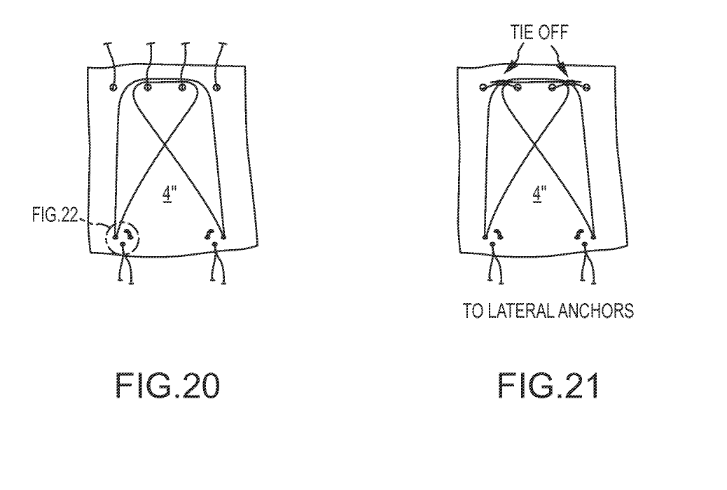

FIGS. 18-22 illustrate another process for creating load sharing with a suture and patch assembly, according to embodiments of the present invention. Four suture ends S6, S7, S8, S9 may be passed through patch 4″, as shown in FIG. 18. This may include two pairs of the ends of two sutures, rather than four separate sutures, according to embodiments of the present invention. Two free sutures S3, S4 may be woven around suture ends S6, S7, S8, and S9 as shown in FIG. 19, then one of the sutures (e.g. S3) may be crossed and the free suture ends passed back through the patch 4″ with self-reinforcing stitches as shown in FIG. 21. The suture end S6 may be tied to suture end S7 and suture end S8 may be tied to suture end S9, as shown in FIG. 21, which forms loops that act similarly to loops 112, 114 described above, according to embodiments of the present invention. The free lateral ends of the patch sutures may be attached to bone anchors and/or tied to bone tunnel sutures as described above, according to embodiments of the present invention. FIG. 22 illustrates an enlarged view of a type of self-reinforcing stitch, a Modified Mason-Allen stitch, according to embodiments of the present invention.

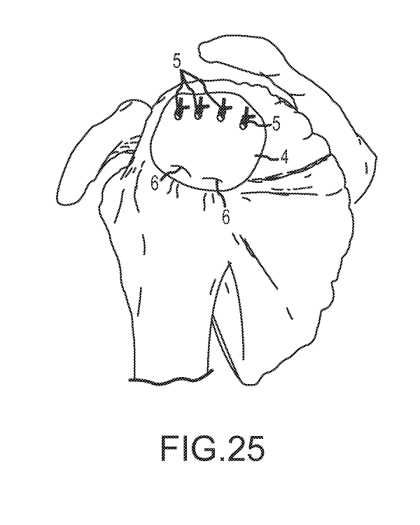

FIGS. 23 and 24 illustrate an alternative self-reinforcing stitch, in which the free lateral ends 116, 118 are brought back above the patch 4 and looped underneath the sutures on the top of patch 4, according to embodiments of the present invention. For example, free end 116 is inserted under suture ends 122 and 124, and free end 118 is inserted under suture ends 124 and 128, as shown in FIGS. 23 and 24, according to embodiments of the present invention.

According to embodiments of the present invention, other repairs may be conducted to the tendon, for example the rotator cuff 20, in addition to the implantation of patch 4, 4′, 4″. For example, the repair of FIG. 4 may be implemented to bring together the damaged edges of the rotator cuff 20 and to put some tension on the rotator cuff 20 for healing, then the patch 4, 4′, 4″ may be applied over the rotator cuff 20 repair and tensioned to absorb some of the load applied across the tendon 20, according to embodiments of the present invention. The patch 4 may be configured to be absorbed into the rotator cuff 20 as tissue ingrowth occurs and as rotator cuff 20 heals, which gradually and naturally shifts the full load back to the rotator cuff 20, according to embodiments of the present invention.

Embodiments of the present invention permit load sharing which is not possible with existing patch repairs of tissue. The suture techniques which provide load sharing of the suture and the graft assembly with the tendon involve a construct which leads to both the suture and the graft sharing loads. This construct results from weaving suture into the patch or graft which, in combination with anchors or other attachment mechanisms, shares the load with the tendon. Embodiments of the present invention also permit use of a suture configuration to grasp the patch or graft (e.g. medial to lateral) and allow the surgeon to tension the patch or graft independently of the tendon and therefore create load sharing with the tendon. When used with knotless suture fixation bone anchors, the patch or graft and suture assembly may be tensioned in a unique way, because the unique tensioning ability of knotless anchors (e.g. a ring cinch type anchor such as the PITON™ available from Tornier®) permits the surgeon to maneuver the patch and suture assembly in new ways to customize the tension. Such anchors allow a suture loop configuration medially that can be cinched down with a cinch anchor, which is not possible with other existing anchors. Such a medial suture loop permits one to pass multiple sutures or stitches through them and then to form a suture bridge over the patch or graft, according to embodiments of the present invention. This, in turn, creates an ability to have the patch and suture assembly load share with the tendon due to the ability to independently tension the patch and suture assembly. Finally, as described above, the use of such a patch and suture assembly with transosseous bone tunnels such as those formed with an ArthroTunneler™ device available from Tornier® also permits arthroscopic tensioning of the patch or graft (and load sharing—with knot configurations) without the need for anchors. Other existing arthroscopic systems are unable to permit such procedures.

Although FIGS. 6-15 illustrate embodiments of methods which use two continuous bone tunnels, other methods may use one bone tunnel, or three, or four, or more bone tunnels, according to embodiments of the present invention.

FIG. 25 illustrates a patch applied with an alternative technique for arthroscopic repair of a rotator cuff tear, according to embodiments of the present invention. FIG. 25 illustrates a patch 4 applied to a shoulder. Patch 4 may be applied arthroscopically over a rotator cuff repair, for example the repair shown in FIG. 4. After the rotator cuff is repaired, two bone anchors may be implanted into the bone under the tendon. Each of the two bone anchors may include two sutures, for example two sutures folded around an eyelet in the bone anchor, such that the two folded sutures form four free suture tails for each bone anchor. These medial anchors (not shown) may be placed, for example, one between the first two knot stacks 5 on FIG. 25 and the other between the last two knot stacks 5 on FIG. 25, according to embodiments of the present invention. Two of the suture tails from the first anchor may be inserted through the rotator cuff tissue and then through the patch 4, and then tied off in a knot 5 over the patch 4 as shown in FIG. 25. Another two of the suture tails from the first anchor may be inserted through the rotator cuff tissue and then through the patch 4, and then tied off in another knot 5 over the patch 4. Then, two of the suture tails from the second bone anchor may be inserted through the rotator cuff tissue and then through patch 4, then tied off in another knot 5 over the patch 4. Finally, the last two suture tails from the second bone anchor may be inserted through the rotator cuff tissue and then through patch 4, then tied off in another knot 5 over the patch.

As such, each of the four knot stacks 5 in FIG. 25 is formed by a pair of suture tails out of four suture tails per anchor, according to embodiments of the present invention. Although bone anchors with eyelets, or with a tie-off configuration are described, other types of bone anchors may be used medially under the tendon. Each pair of the suture tails is inserted through the tendon and through the patch 4 or graft at a different location, in order to further distribute the load. The greater the number of independent sutures and suture attachment locations, the higher the load-to-failure ratio, according to embodiments of the present invention. For example, using eight independent suture tails as shown in FIG. 25 as opposed to four may increase the load-to-failure ratio by two or more.

Next, a free suture 6 is passed through the patch 4 in a mattress or other self-reinforcing type configuration at or near the lateral edge of the patch 4, according to embodiments of the present invention. This self-reinforcing configuration includes passing the suture through the patch 4 and then back through the patch in either direction (top to bottom or bottom to top) in order to increase the suture pullout resistance and distribute the load across the patch 4, according to embodiments of the present invention. The more a suture end is passed through a patch 4 or graft and then back through it again, the more of a “weave” which is created, and the more complex of a “grab” is created, the more difficult it becomes to rip out the suture, and the more load is transmitted from suture to patch, according to embodiments of the present invention.

A tensional-type bone anchor may be placed laterally (or distally) to the lateral (or distal) edge of the rotator cuff, and the free ends of the suture 6, which has already been stitched through patch 4 in a self-reinforcing manner, may be placed through the tensional-type bone anchor (not shown). The tension on the tails of suture 6 may then be adjusted in order to independently adjust the tension on the patch 4. This same technique may be performed twice or more at the lateral edge of the patch 4, according to embodiments of the present invention, as shown in FIG. 25. Alternatively, tied anchors or other types of bone anchors may be used laterally to the rotator cuff to receive the free pairs of ends of sutures 6; in such cases, the desired tension may be placed on the sutures 6 (and hence patch 4) prior to or during the final fixation to the bone anchor, according to embodiments of the present invention.

According to some embodiments of the present invention, the medial anchoring of the patch 4 (e.g. at knot stacks 5) and/or the medial placement of the suture anchors, may be located medial of the medial edge of the rotator cuff repair (not visible in FIG. 25), so that the repaired rotator cuff tissue is completely bypassed with the load sharing of the patch 4, according to embodiments of the present invention. The lateral anchors may also be placed laterally of the edge of the rotator cuff, and/or the rotator cuff repair, to achieve the same goal, according to embodiments of the present invention. In this way, the load will be taken up by the patch 4 or graft, rather than the actual tendon.

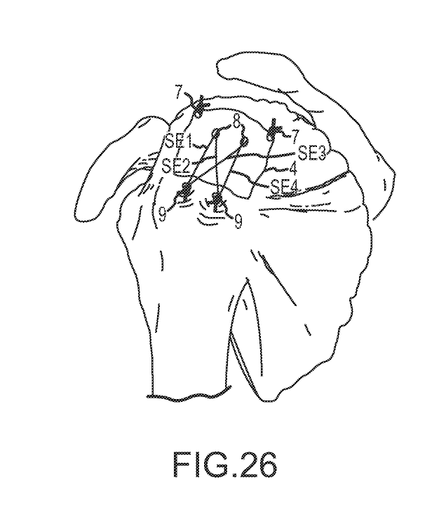

FIG. 26 illustrates a patch 4 applied with yet another alternative technique for arthroscopic repair of a rotator cuff tear, according to embodiments of the present invention. FIG. 26 illustrates a patch 4 applied to a shoulder. Patch 4 may be applied arthroscopically over a rotator cuff repair, for example the repair shown in FIG. 4. After the rotator cuff is repaired, two bone anchors may be implanted into the bone under the tendon, similarly to the technique described with respect to FIG. 25. However, instead of the two sutures per bone anchor as described in FIG. 25, the two bone anchors in the technique of FIG. 26 may each have only one suture, or two free suture tails for each bone anchor, according to embodiments of the present invention. The two free suture ends from one bone anchor may be passed through the tendon, then through the patch 4, and then tied off above the patch 4 as shown at knot stack 8. Likewise, the two free suture ends from the other bone anchor may be passed through the tendon and through the patch 4 and then tied off above the patch 4 as shown at knot stack 8, according to embodiments of the present invention. Then one free suture end SE1 from one knot stack is joined with another free suture end SE3 from another knot stack 8 and they are both are fixated at a common fixation point (e.g. inserted through a bone anchor 9), according to embodiments of the present invention. Likewise, another free suture end SE2 from one knot stack 8 is joined with another free suture end SE4 from the other knot stack 8 and both are fixated at a common fixation point (e.g. inserted through a bone anchor 9), with suture ends SE2 and SE3 forming a crossing pattern over the top of the patch 4, according to embodiments of the present invention.

The suture anchors 9 are placed laterally (or distally) of the lateral (or distal) edge of the rotator cuff and laterally (or distally) of the patch 4, according to embodiments of the present invention. This permits load sharing to be achieved across the patch 4, and also prevents “bunching up” of the patch distally, according to embodiments of the present invention. The suture anchors 9 may be a tensionable-type suture anchor 9, or may alternatively be tie-off suture anchors, according to embodiments of the present invention. The medial suture anchors (not shown) which connect to the sutures from which knot stacks 8 are formed are separated from one another, and knot stacks 8 are at different locations in the patch 4, according to embodiments of the present invention.

The knot stacks indicated at 7 are each formed by a free strand of suture which is passed through the rotator cuff and through the patch 4 and tied to itself, for example with one free end of the suture extending up through the patch 4 and another extending up through the cuff and then tied together, according to embodiments of the present invention. These sutures/knot stacks at 7 are thus not anchored to the bone, and are placed at different locations with respect to the knot stacks 8. For example, the sutures at 7 are placed medially of the fixation points 8, such that they are not in the same plane, thus encouraging load sharing across the patch 4, because fixation points 7 are placed medial to the suture line of 8, according to embodiments of the present invention. As such, a suture and patch construct is formed with anchors directly under the cuff medially, and anchors placed laterally to the lateral edges of the tendon and the patch 4, according to embodiments of the present invention.

Although various features and characteristics are described herein with respect to certain embodiments or in certain combinations, such features and/or characteristics may be used or combined with other embodiments or other features even if such combinations are not expressly described.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method for implementing load sharing among a tissue to be repaired and a patch, the method comprising:
arthroscopically forming a first bone tunnel, the first bone tunnel continuous from a first end to a second end;
arthroscopically forming a second bone tunnel, the second bone tunnel continuous from a first end to a second end;
arthroscopically inserting a first suture loop through the first bone tunnel into the first end and out of the second end, the first suture loop formed of a first suture;
arthroscopically inserting a second suture loop through the second bone tunnel into the first end and out of the second end, the second suture loop formed of a second suture;
passing the first suture loop through a patch, wherein the patch is a synthetic or biologic biocompatible tissue repair patch;
passing the second suture loop through the patch; and
passing a third suture through the first and second suture loops.

2. The method of claim 1, further comprising:
passing a first end of the third suture through the patch; and
passing a second end of the third suture through the patch.

3. The method of claim 2, further comprising crossing the first and second ends of the third suture over the patch before passing the first and second ends of the third suture through the patch.

4. The method of claim 1, further comprising:
passing a fourth suture through the first and second suture loops.

5. The method of claim 4, further comprising:
passing a first end of the third suture through the patch;
passing a second end of the third suture through the patch;
passing a first end of the fourth suture through the patch; and
passing a second end of the fourth suture through the patch.

6. The method of claim 5, further comprising:
passing the first end of the third suture through the patch through a first hole; and
passing the second end of the third suture through the patch through a second hole separated from the first hole.

7. The method of claim 6, further comprising:
passing the first end of the fourth suture through the first hole; and
passing the second end of the fourth suture through the second hole.

8. The method of claim 7, further comprising:
crossing the first and second ends of the fourth suture before passing the first end of the fourth suture through the first hole and the second end of the fourth suture through the second hole.

9. The method of claim 8, further comprising:
applying a first self-reinforcing stitch through the patch with the first end of the third suture and the first end of the fourth suture; and
applying a second self-reinforcing stitch through the patch with the second end of the third suture and the second end of the fourth suture.

10. The method of claim 9, further comprising:
tensioning the patch by tensioning the first and second ends of the third and fourth sutures.

11. The method of claim 12, further comprising tying the first suture to the second suture across the bone to form a suture bridge.

12. The method of claim 10, further comprising:
tying the first ends of the third and fourth sutures to the suture bridge; and
tying the second ends of the third and fourth sutures to the suture bridge.

13. A method for implementing load sharing among a tissue to be repaired and a patch, the method comprising:
passing, arthroscopically, a first suture loop of a first suture through tissue to be repaired;
passing the first suture loop through a patch, wherein the patch is a synthetic or biologic biocompatible surgical repair patch;
passing, arthroscopically, a second suture loop of a second suture through the tissue;
passing the second suture loop through the patch;
passing a third suture through the first and second suture loops;
passing a first end of the third suture through the patch;
passing a second end of the third suture through the patch;
attaching, arthroscopically, the first suture to a bone under the tissue;
attaching, arthroscopically, the second suture to the bone under the tissue;
tensioning the third suture and the first and second suture loops over the patch; and
anchoring the first and second ends of the third suture to the bone.

14. The method of claim 13, further comprising:
passing a fourth suture through the first and second suture loops;
passing a first end of the fourth suture through the patch;
passing a second end of the fourth suture through the patch; and
attaching the first and second ends of the fourth suture to the bone.

15. The method of claim 13, wherein anchoring the first and second ends of the third suture to the bone comprises attaching at least one of the first and second ends of the third suture to a ring cinch suture anchor, the method further comprising:
adjusting a tension of the at least one of the first and second ends of the third suture independently of a tension of the tissue.

16. The method of claim 15, further comprising:
making a first tension adjustment to the at least one of the first and second ends of the third suture;
observing the tension of the tissue; and
making a second tension adjustment to the at least one of the first and second ends of the third suture based on the observation.

17. A method for implementing load sharing among rotator cuff tissue to be repaired and a patch and suture assembly, the method comprising:
forming the patch and suture assembly from a patch and suture to permit tension applied to the suture to be transmitted through the patch;
anchoring the patch and suture assembly medially through the rotator cuff tissue;
adjusting a tension of the patch and suture assembly independent of a tension of the rotator cuff tissue; and
anchoring the patch and suture assembly laterally at the adjusted tension.

18. The method of claim 17, wherein anchoring the patch and suture assembly laterally comprises installing a tensionable suture anchor, and wherein adjusting the tension of the patch and suture assembly comprises pulling the suture through the tensionable suture anchor.

19. The method of claim 17, wherein anchoring the patch and suture assembly medially and laterally comprises tying the patch and suture assembly to one or more transosseous bone tunnels.

20. The method of claim 19, wherein anchoring the patch and suture assembly comprises tying the patch and suture assembly to one or more transosseous bone tunnels without the use of bone anchors.
